(19) United States
(12) Patent Application Publication
(10) Pub. No.: US 2019/0267134 A1
(43) Pub. Date: Aug. 29, 2019

(54) System and Method for the Specialized Delivery of Telemedicine Services

(71) Applicant: Stanley G. Van Meter, II, Lake Mary, FL (US)

(72) Inventor: Stanley G. Van Meter, II, Lake Mary, FL (US)

(21) Appl. No.: 16/288,078

(22) Filed: Feb. 27, 2019

Related U.S. Application Data
(60) Provisional application No. 62/636,029, filed on Feb. 27, 2018.

(51) Int. Cl. G16H 40/20; G16H 10/60; G06Q 10/06; G06Q 10/10

(52) U.S. Cl. CPC G16H 40/20; G16H 10/60; G16H 40/63; G06Q 10/10; G06Q 10/06311

(57) ABSTRACT

The present invention is a computer implemented platform for connecting members/patients with medical health provider and professionals. At various times during the registration and exam requesting process, the platform can recommend medical service providers and practice groups based on a user's gender or simply provide them with the first available provider. The user can set up the platform to limit services to be provided from a preferred gender of medical professional set by the user and the system can offer or suggest services to be provided by a certain gender based on intake information such as the sex of the user requesting an exam, the type of exam, user preferences, or any number of other factors or considerations such as medical records, past exams, exam notes, or any criteria or combination of criteria or information provided to the platform by the user.

### FEDERALLY SPONSORED RESEARCH

Not Applicable

### SEQUENCE LISTING OR PROGRAM

Not Applicable

### TECHNICAL FIELD OF THE INVENTION

The present invention relates in general to the remote delivery of medical services. More specifically, the present invention relates to the remote delivery of specialized medical services based using smartphones or other mobile devices, apps., and electronic records, where the patients information and medical records are used to offer, suggest, and provide specialized medical services over a platform connecting patients with medical service providers and professionals.

### BACKGROUND OF THE INVENTION

Telemedicine is the use of telecommunication and information technology to provide clinical health care from a distance. It has been used to overcome distance barriers and to improve access to medical services that would often not be consistently available in distant rural communities. It is also used to save lives in critical care and emergency situations.

Although there were distant precursors to telemedicine, it is essentially a product of 20th century telecommunication and information technologies. These technologies permit communications between patient and medical staff with both convenience and fidelity, as well as the transmission of medical, imaging and health informatics data from one site to another.

Early forms of telemedicine achieved with telephone and radio have been supplemented with video telephony, advanced diagnostic methods supported by distributed client/server applications, and additionally with tele medical devices to support in-home care.

Telemedicine can be beneficial to patients in isolated communities and remote regions, who can receive care from doctors or specialists far away without the patient having to travel to visit them. Recent developments in mobile collaboration technology can allow healthcare professionals in multiple locations to share information and discuss patient issues as if they were in the same place. Remote patient monitoring through mobile technology can reduce the need for outpatient visits and enable remote prescription verification and drug administration oversight, potentially significantly reducing the overall cost of medical care. Telemedicine can also facilitate medical education by allowing workers to observe experts in their fields and share best practices more easily.

The downsides of telemedicine include the cost of telecommunication and data management equipment and of technical training for medical personnel who will employ it, and an increased risk that protected health information may be compromised through electronic storage and transmission.

Additionally, potentially poor quality of transmitted records, such as images or patient progress reports, and decreased access to relevant clinical information are quality assurance risks that can compromise the quality and continuity of patient care for the reporting doctor. Other obstacles to the implementation of telemedicine include unclear legal regulation for some tele medical practices and difficulty claiming reimbursement from insurers or government programs in some fields.

### SUMMARY OF THE INVENTION

The present invention is a computer implemented platform for connecting members/patients with medical health provider and professionals.

The first step in the new user methodology flow where an introduction video is played for the user/member/patient first to explain the functionality of the platform and how the process work before requesting the user to enter their, phone number and a confirmation code as a result of an SMS text sent to the user's entered phone number.

Next the user enters their user information such as name, address, email, and password. After establishing an account and entering all account information, the user is presented with a chief complaint series of screens for providing medical intake information that will be collected, processed, and provided to a medical professional as part of the platforms connection process.

First, the user is present with a list of common complaints from which a selection can be made, or alternatively, they can search for complaint or reason. Once found, the user will select the complaint or reason and then is provided with an opportunity to upload images, which may be helpful to their condition and a diagnosis.

Before submitting the information, the user must agree to a variety of terms, such as HIPPA compliance terms, platform/application terms, provider terms, and video chat terms. HIPAA and T&C for the platform/application are typically only asked the first time unless there is a change to them, and they can be viewed individually at any time while a user is logged into the platform/application.

At various times during the registration and exam requesting process, the platform can recommend medical service providers and practice groups based on a user's gender or simply provide them with the first available provider. The user can set up the platform to limit services to be provided from a preferred gender of medical professional set by the user and the system can offer or suggest services to be provided by a certain gender based on intake information such as the sex of the user requesting an exam, the type of exam, user preferences, or any number of other factors or considerations based on intake information, medical records, past exams, exam notes, or any criteria or combination of criteria or information provided to the platform by the user.

There are three options a user has for selecting a pharmacy, first using their devices GPS and a map, with potential pharmacies noted as pins, by entering a zip code to locate the closest pharmacies if no GPS or map location is available, and selecting from a list based on either GPS location or zip codes inputs.

The process of picking a plan and adding family members to the account on the platform/application are illustrated. The user is provided with a single or family plan option, which includes information on what is covered by each. Once a plan is selected, then the names of family members can be added, as well as an address and subscription service. Once a plan is selected and all information entered, the final payment screen is displayed where the user will add credit card or other payment information.

The user's request exam information is displayed and presented for review while the user waits for tele-connection with medical services professional. While waiting for a medical services provider, the user an complete a medical history intake form or update any such form connected and stored across the platform with an electronic medical recorders provider. A user is displayed a plurality of screens, asking them to enter yes/no questions with respect to conditions, allergies, and other relevant questions. Once the intake of additional medical information has been completed, the user is displayed a waiting room screen notifying them of their wait time and any additional pertinent information.

A video call within the app is display to a user if required by state law, which is then followed up by a SMS text notification when any prescriptions prescribed as a result of the appointment and have been filled by the selected pharmacy.

Next a user selects the family member that is to be seen by a medical service provider. The user either selects a chief complaint from a list of menus or by entering an unspecified complaint and can upload one or more images if/as necessary for assistance in being evaluated. The user is then offer the choice to select or confirm an existing pharmacy from either a map display or a list display of potential pharmacy options based on location.

A biometric login is presented to the user while the system searches for and ques an available doctor to fulfil the request. Biometric confirmation of the requested exam can be provided either with touch identification or a facial identification. A standard phone number and password login may also be presented for verification, along with standard password recovery options.

Finally, while waiting for a doctor, the user can review all their previously entered information and selections to make changes or edits. Once all previously information is confirmed and final, a static display is presented to the user while waiting for a medical professional.

### Definitions

“Application software” or “software” is a set of one or more programs designed to carry out operations for a specific application. Application software cannot run on itself but is dependent on system software to execute. Examples of application software include MS Word, MS Excel, a console game, a library management system, a spreadsheet system etc. The term is used to distinguish such software from another type of computer program referred to as system software, which manages and integrates a computer's capabilities but does not directly perform tasks that benefit the user. The system software serves the application, which in turn serves the user.

The term “app” is a shortening of the term “application software”. “Apps” are usually available through application distribution platforms, which began appearing in 2008 and are typically operated by the owner of the mobile operating system. Some apps are free, while others must be bought. Usually, they are downloaded from the platform to a target device, but sometimes they can be downloaded to laptops or desktop computers.

“Electronic Mobile Device” is defined as any computer, phone, or computing device that is comprised of a battery, display, circuit board, and processor that is capable of processing or executing software. Examples of electronic mobile devices are smartphones, laptop computers, and table PCs.

The Global Positioning System (GPS) is a space-based navigation system that provides location and time information in all weather conditions, anywhere on or near the Earth where there is an unobstructed line of sight to four or more GPS satellites.

“GUI”. In computing, a graphical user interface (GUI) sometimes pronounced “gooey” (or “gee-you-eye”)) is a type of interface that allows users to interact with electronic devices through graphical icons and visual indicators such as secondary notation, as opposed to text-based interfaces, typed command labels or text navigation.

“Telemedicine” is has several industry definitions, such as the definition given by the World Health Organization including all aspects of healthcare including preventive care. The American Telemedicine Association uses the terms telemedicine and telehealth interchangeably, although it acknowledges that telehealth is sometimes used more broadly for remote health not involving active clinical treatments. “eHealth” is another related term, used particularly in the U.K. and Europe, as an umbrella term that includes telehealth, electronic medical records, and other components of health information technology

A “mobile app” is a computer program designed to run on smartphones, tablet computers and other mobile devices, which the Applicant/Inventor refers to generically as “a computing device”, which is not intended to be all inclusive of all computers and mobile devices that are capable of executing software applications.

A “smartphone” (or smart phone) is a mobile phone with more advanced computing capability and connectivity than basic feature phones. Smartphones typically include the features of a phone with those of another popular consumer device, such as a personal digital assistant, a media player, a digital camera, and/or a GPS navigation unit. Later smartphones include all of those plus the features of a touchscreen computer, including web browsing, wideband network radio (e.g. LTE), Wi-Fi, 3rd-party apps, motion sensor and mobile payment.

A “web application” or “web app” is any application software that runs in a web browser and is created in a browser-supported programming language (such as the combination of JavaScript, HTML and CSS) and relies on a web browser to render the application.

### BRIEF DESCRIPTION OF THE DRAWINGS

The accompanying drawings, which are incorporated herein form a part of the specification, illustrate the present invention and, together with the description, further explain the principles of the invention and to enable a person skilled in the pertinent art to make and use the invention.

FIG. 1 illustrates the new user application flow for registering and using the platform for the first time.

FIGS. 2a and 2b illustrate the login and confirmation screens of the mobile application.

FIGS. 3a, 3b, 3c, and 3d illustrate the reason for exam entry screens displayed by the mobile application.

FIGS. 4a, 4b, and 4c illustrate the pharmacy selection method embodied by the mobile application.

FIGS. 5a, 5b, and 5c illustrate the plan selection method and entry of covered family members embodied by the mobile application.

FIG. 6 illustrates the request an exam method embodied by the mobile application.

FIGS. 7a, 7b, and 7c illustrate the medical history information intake method embodied by the mobile application.

FIG. 8 illustrates the request an exam method embodied by the mobile application.

FIGS. 9a and 9b illustrate the video chat method embodied by the mobile application.

FIG. 10 illustrates the prescription ready notice generated by the mobile application.

FIG. 11 illustrates the application flow for an existing user returning to the platform to set up a new exam with a medical provider.

FIGS. 12a and 12b illustrate the request an exam method embodied by the mobile application.

FIG. 13 illustrates the application flow for an existing user returning to the platform to review past exams and doctor notes.

FIGS. 14a and 14b illustrate the home screen and an member's dashboard as present to a user generated by the mobile application.

FIG. 15 illustrates a login screen for a returning user as generated by the mobile application.

FIG. 16 illustrates a password recovery screen for a returning user as generated by the mobile application.

FIG. 17 illustrates a display screen for reviewing past exams and making document notes as generated by the mobile application.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figure, it is possible to see the various major elements constituting the apparatus of the present invention.

Now referring to the Figures, the embodiment of the system and method for providing a computer implemented platform for connecting members/patients with medical health provider and professionals is illustrated. The method of the present invention is embodied by a software program containing executable instruction of the method and/or process claimed by the present invention. The software program embodying the present invention is executable on a particular machine or apparatus. “Particular machine” or “apparatus” is defined as a desktop computer, laptop computer, personal data assistance (PDA), IPAD, tablets, table computers, IPHONE, mobile phone, smart phone, or any other equivalent electronic device which is capable of running a set of executable instructions embodied by software and providing a display of the result of those instructions. These electronic devices used to define a particular machine or apparatus function tie the method of the present invention to a particular machine or apparatus.

The present invention is a computer implemented platform for connecting members/patients with medical health provider and professionals.

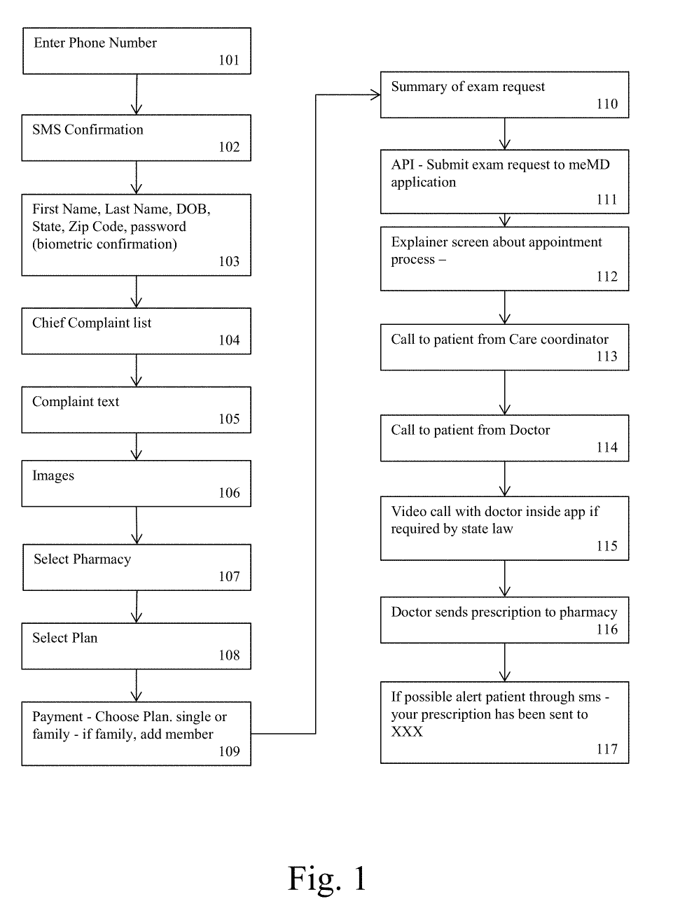

Now referring to FIGS. 1-10, the application (app.) flow for a new user becoming a member of the platform is illustrated. FIG. 1 illustrates the information collected as part of the new user flow being: phone number 101, SMS confirmation 102, full name, date of birth, zip code, and password 103, a chief complaint 104, complaint text 105, images 106, a pharmacy selection 107, a medical plan selection 109, payment option, summary of exam request 110, API—Submit exam request to me MD application 111, appointment explainer screen display 112, a call to patient from a care coordinator 113, a call to the patient from a doctor or other medical professional 114, a video call occurring inside the “app” on the platform 115, if required by state law, medical professional submitting a prescription to a pharmacy 116, if appropriate, a potential text alter to patient through SMS confirm the prescription being sent to a selected pharmacy 117.

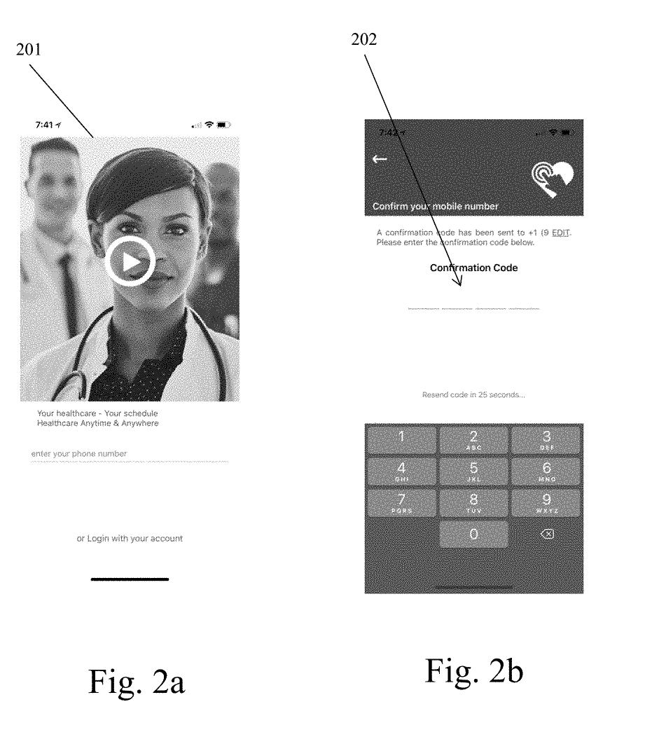

FIGS. 2a and 2b illustrate the first step in the new user methodology flow where an introduction video 201 is played for the user/member/patient first to explain the functionality of the platform and how the process work before requesting the user to enter their, phone number and a confirmation code 202 as a result of an SMS text sent to the user's entered phone number.

Next the user enters their user information as described above, such as name address, email, and password. After establishing an account and entering all account information, the user is presented with a chief complaint series of screens for providing medical intake information that will be collected, processed, and provided to a medical professional as part of the platforms connection process.

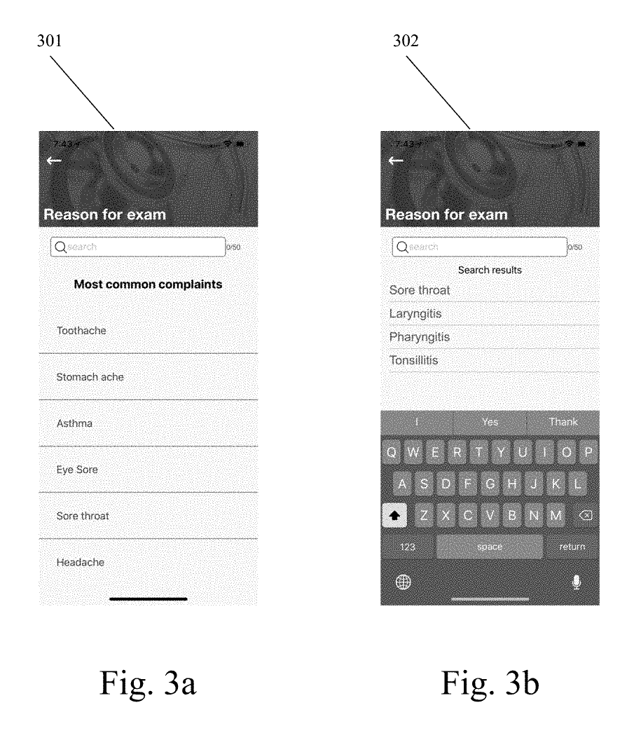

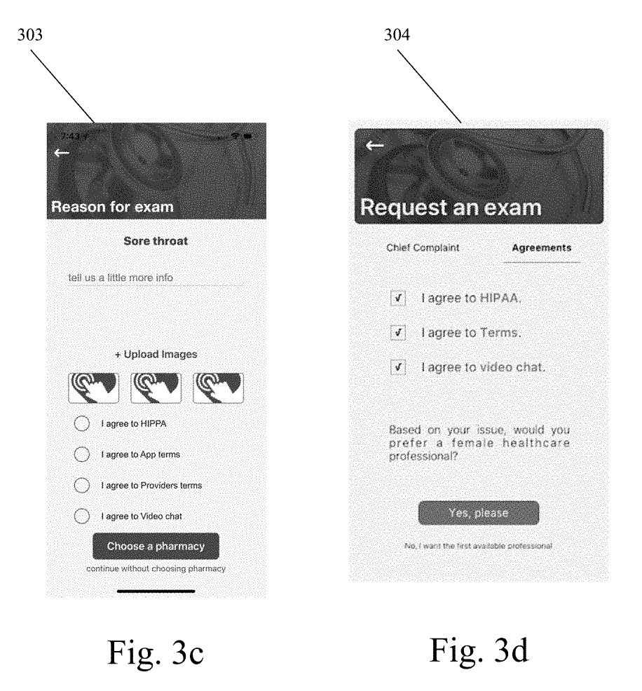

FIGS. 3a, 3b, 3c, and 3d illustrate a four screen display process where a user enters the reasons for seeking an examination and consultation with a medical professional. First, the user is presented with a list of common complaints from which a selection can be made 301, or alternatively, they can search for complaint or reason 302. Once found, the user will select the complaint or reason 303 and then is provided with an opportunity to upload images, which may be helpful to their condition and a diagnosis. Before submitting the information, the user must agree to a variety of terms, such as HIPPA compliance terms, platform/application terms, provider terms, and video chat terms 304. HIPAA and T&C for the platform/application are typically only asked the first time unless there is a change to them, and they can be viewed individually at any time while a user is logged into the platform/application.

At various times during the registration and exam requesting process, as shown in FIG. 3, the platform can recommend medical service providers and practice groups based on a user's gender or simply provide them with the first available provider. The user can set up the platform to limit services to be provided from a preferred gender of medical professional set by the user and the system can offer or suggest services to be provided by a certain gender based on intake information such as the sex of the user requesting an exam, the type of exam, user preferences, or any number of other factors or considerations based on intake information, medical records, past exams, exam notes, or any criteria or combination of criteria or information provided to the platform by the user.

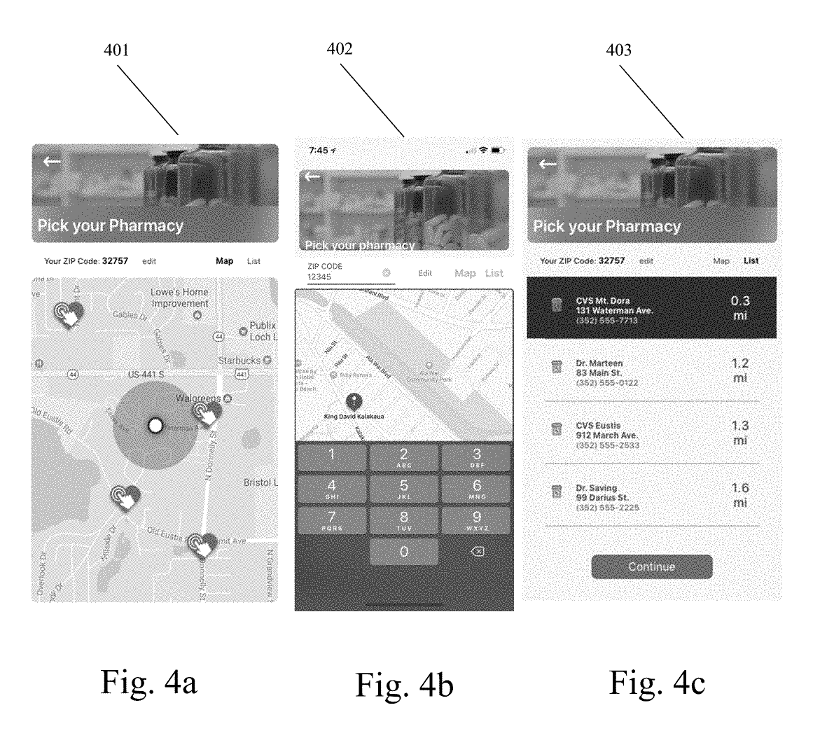

FIGS. 4a, 4b, and 4c illustrate the three options a user has for selecting a pharmacy, first using their devices GPS and a map 401, with potential pharmacies noted as pins, by entering a zip code to locate the closest pharmacies if no GPS or map location is available 402, and selecting from a list based on either GPS location or zip codes inputs 403.

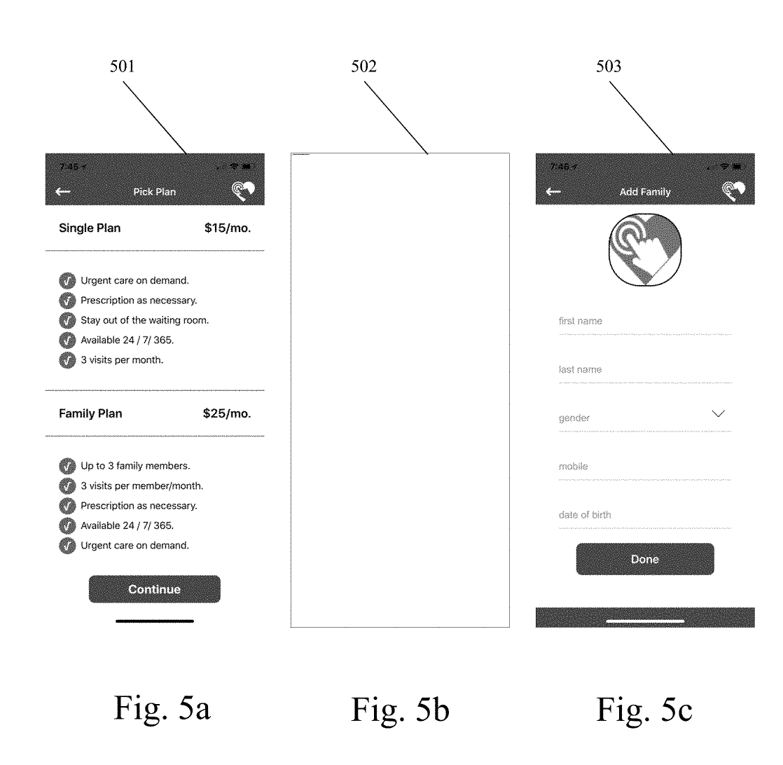

Now referring to FIGS. 5a, 5b, and 5c, the process of picking a plan and adding family members to the account on the platform/application are illustrated. The user is provided with a single or family plan option 501, which includes information on what is covered by each. Once a plan is selected, then the names of family members can be added, as well as an address and subscription service 502. Once a plan is selected and all information entered, the final payment screen is displayed where the user will add credit card or other payment information 503.

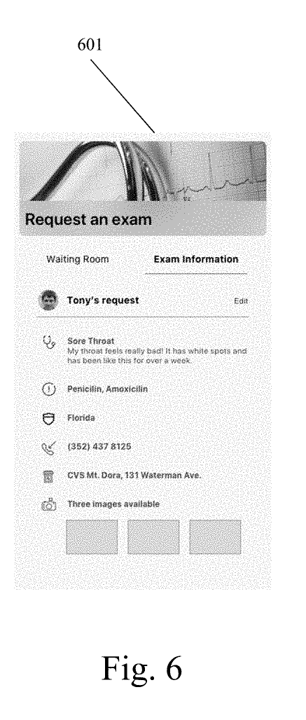

In FIG. 6, the user's request exam information is displayed and presented for review while the user waits for tele-connection with medical services professional 601.

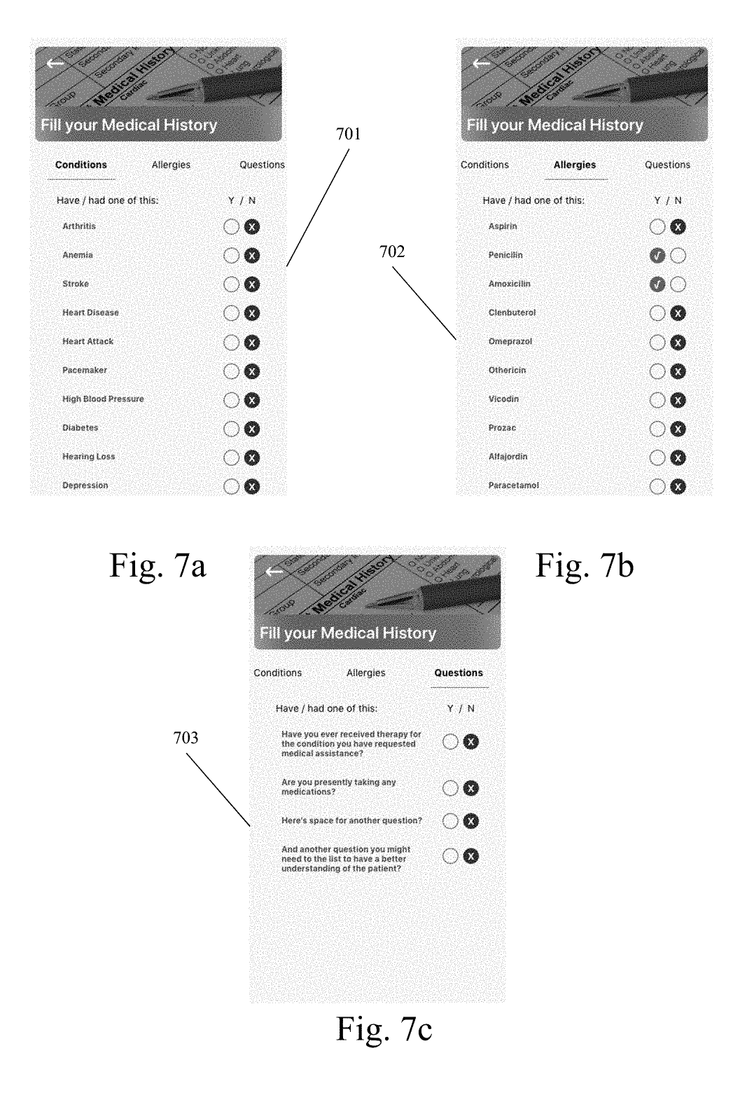

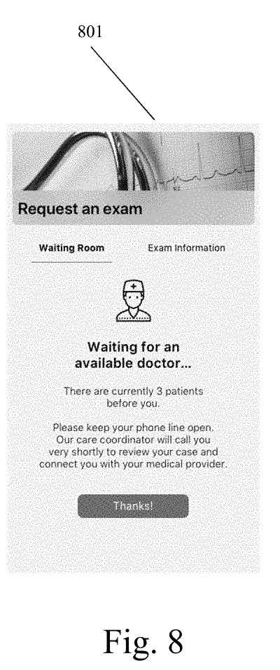

While waiting for a medical services provider, the user an complete a medical history intake form or update any such form connected and stored across the platform with an electronic medical recorders provider. A user is displayed a plurality of screens as shown in FIGS. 7a, 7b, and 7c, asking them to enter yes/no questions with respect to conditions, allergies, and other relevant questions 701, 702, and 703. Once the intake of additional medical information has been completed, the user is displayed a waiting room screen 801 as shown in FIG. 8, notifying them of their wait time and any additional pertinent information.

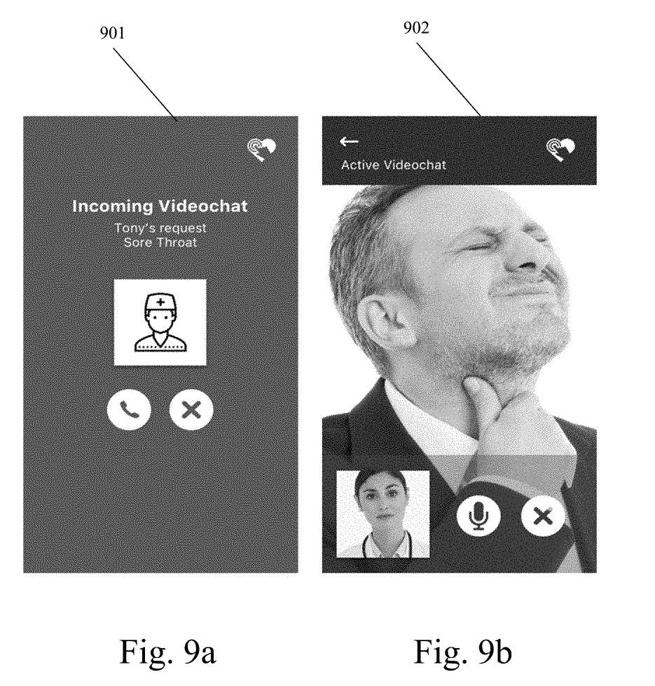

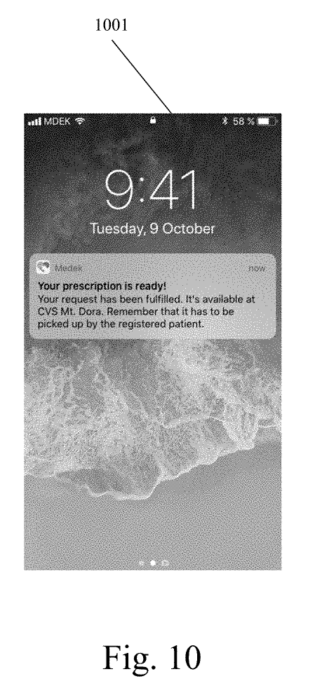

FIGS. 9a, 9b, and 10 illustrate the new user flow, where a video call within the app is display to a user if required by state law 901 and 902, which is then followed up by a SMS text notification 1001 when any prescriptions prescribed as a result of the appointment and have been filled by the selected pharmacy.

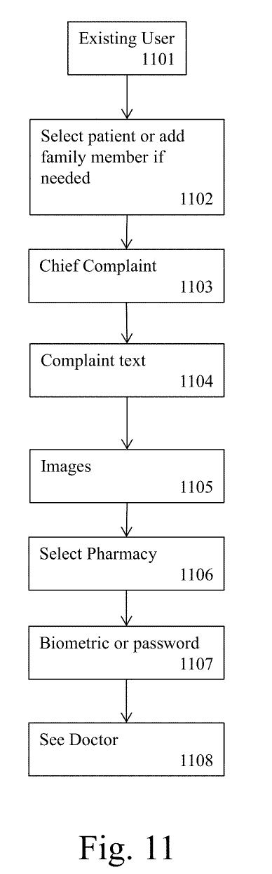

Now referring to the existing user 1101 flow illustrates in FIGS. 11-21, first a user selects the family member that is to be seen by a medical service provider 1102. Next the user either selects a chief complaint from a list of menus 1103 or by entering an unspecified complaint 1104 and can upload one or more images if/as necessary for assistance in being evaluated 1105. The user is then offer the choice to select or confirm an existing pharmacy from either a map display or a list display of potential pharmacy options based on location 1106. Next a biometric login is presented to the user while the system searches for and ques an available doctor to fulfil the request 1107. Biometric confirmation of the requested exam can be provided either with touch identification 1201 or a facial identification 1202. A standard phone number and password login may also be presented for verification 1501, along with standard password recovery options 1601. Finally, while waiting for a doctor, the user can review all their previously entered information and selections to make changes or edits 1402. Once all previously information is confirmed and final, a static display 801 is presented to the user while waiting for a medical professional. Finally, the user sees the doctor or medical professional 1108.

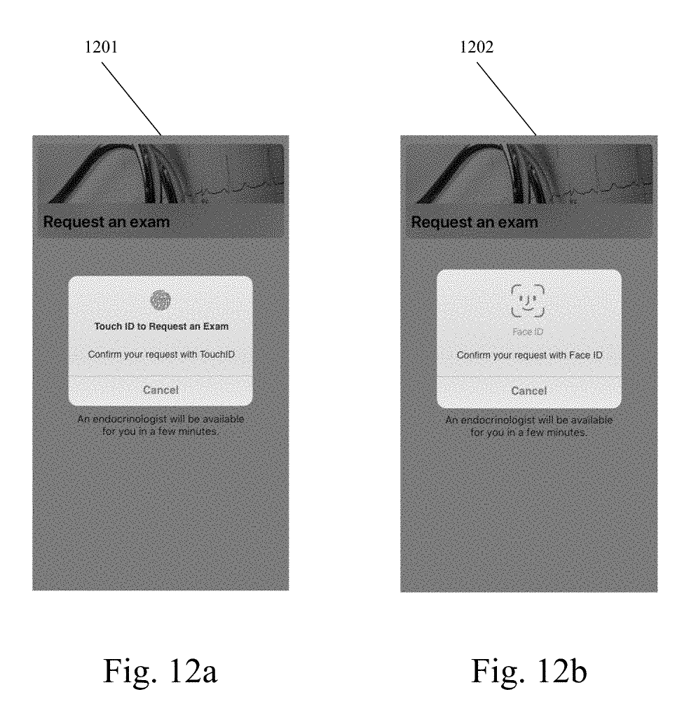

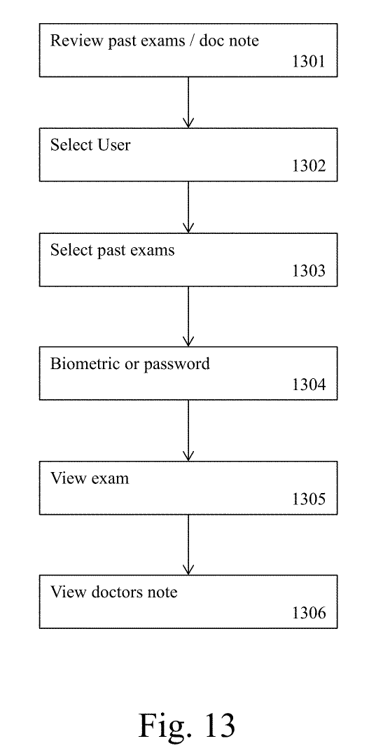

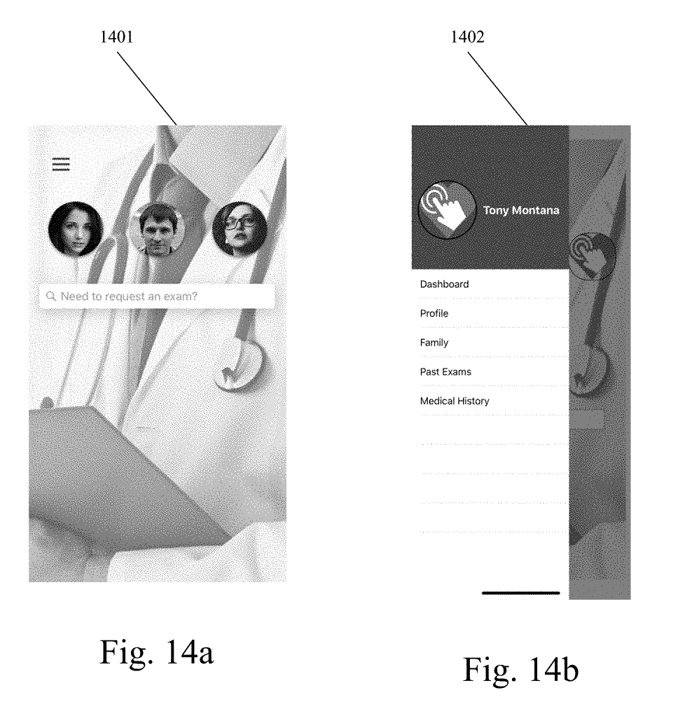

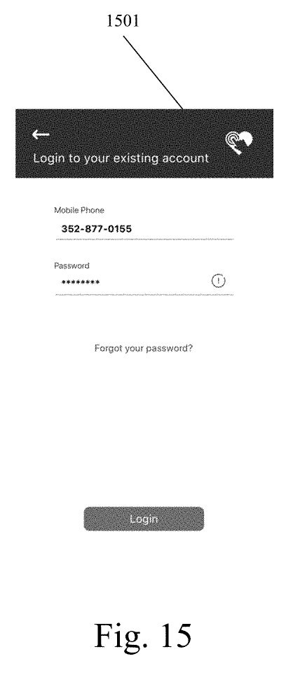

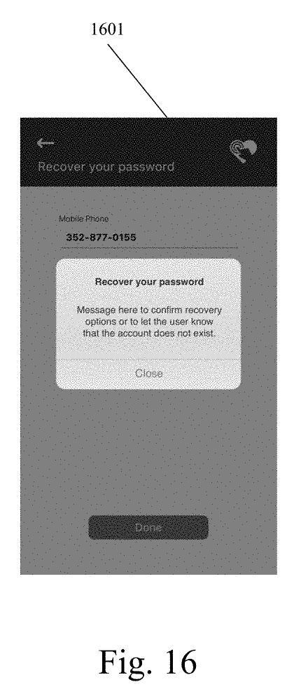

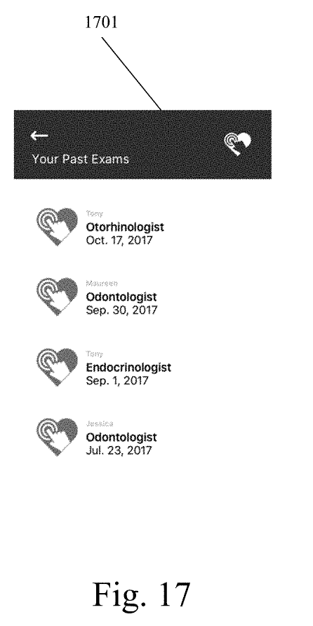

Now referring to FIG. 13 the application flow for reviewing past exams and making document notes is illustrated. While using the platform a user can review past exams or review doctor notes from a past exam 1301. In a first step, the user selects a member of the user's account 1302 to review the selected user's exam records as shown in FIGS. 14a and 14b illustrating a user account screen 1401 and individual member of a user's account dashboard 1402. Again, Biometric confirmation 1304 of the requested exam can be provided either with touch identification 1201 or a facial identification 1202 as shown in FIGS. 12a and 12b. A standard phone number and password login may also be presented for verification, along with standard password recovery options. A standard phone number and password login 1501 may also be presented for verification, along with standard password recovery options 1601 as shown in FIGS. 15 and 16. Finally, a user can simply select from a list of present past exams 1303, the exam 1701 they would like to review as show in FIG. 17. Once selected, the exam summary 1305 and any doctor notes will be displayed to the user 1306.

The system is set to run on a computing device or mobile electronic device. A computing device or mobile electronic device on which the present invention can run would be comprised of a CPU, Hard Disk Drive, Keyboard, Monitor, CPU Main Memory and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The embodiments of the invention in which an exclusive property or privilege is claimed are defined as follows:

1. A computer implemented method for connecting members/patients with medical health provider and professionals, comprising the steps of:
running and executing an application delivering a medical health platform connecting medical professionals and patients on an electronic device;
registering a user for the platform;
collecting new user information;
collecting medical plan information;
setting a plan and payment option;
recording a pharmacy selection;
creating an exam request;
determining a chief complaint;
receiving any uploaded images;
creating a summary of the complaint;
displaying an appointment explainer screen display;
placing a call to patient from a care coordinator;
facilitating a call to the patient from a doctor or other medical professional;

2. The method of claim 1, wherein new user information includes phone number, SMS confirmation, full name, date of birth, zip code, and password.

3. The method of claim 1, wherein a video call occurring inside the application on the platform, if required by state law.

4. The method of claim 1, further comprising the step of submitting a prescription to a selected pharmacy.

5. The method of claim 4, further comprising the step of sending a potential text to the patient through SMS to confirm the prescription being sent to a selected pharmacy.

6. The method of claim 1, further comprising the step of
the first step in the new user methodology flow where an introduction video is played for the user/member/patient first to explain the functionality of the platform and how the process work before requesting the user to enter their, phone number; and
sending a confirmation code as a result of an SMS text sent to the user's entered phone number.

7. The method of claim 6, after establishing an account and entering all account information, further comprising the steps of
presenting the user with a chief complaint series of screens for providing medical intake information; and
collecting, processing, and providing to a medical professional medical intake information received as part of the platform's connection process.

7. The method of claim 1, further comprising the steps of
a three screen display process where a user enters the reasons for seeking an examination and consultation with a medical professional;
the user is present with a list of common complaints from which a selection can be made, or alternatively, they can search for complaint or reason;
the user will select the complaint or reason and then is provided with an opportunity to upload images, which may be helpful to their condition and a diagnosis;
before submitting the information, the user must agree to a variety of terms and conditions.

8. The method of claim 7, wherein the terms and conditions include HIPPA compliance terms, platform/application terms, provider terms, and video chat terms.

9. The method of claim 8, wherein the HIPAA and T&C for the platform/application are typically only asked the first time unless there is a change to them, and they can be viewed individually at any time while a user is logged into the platform/application.

10. The method of claim 6, further comprising the step of
at various times during the registration and exam requesting process the platform can recommend medical service providers and practice groups based on a user's gender or simply provide them with the first available provider.

11. The method of claim 10, wherein
the user can set up the platform to limit services to be provided from a preferred gender of medical professional set by the user and the system can offer or suggest services to be provided by a certain gender based on intake information.

12. The method of claim 11, wherein
Intake information includes the sex of the user requesting an exam, the type of exam, user preferences, or any number of other factors or considerations based on intake information, medical records, past exams, exam notes, or any criteria or combination of criteria or information provided to the platform by the user.

13. The method of claim 1, wherein
the three options a user has for selecting a pharmacy,
first using their devices GPS and a map, with potential pharmacies noted as pins,
by entering a zip code to locate the closest pharmacies if no GPS or map location is available, and
selecting from a list based on either GPS location or zip codes inputs.

14. The method of claim 1, wherein
the user is provided with a single or family plan option, which includes information on what is covered by each;
once a plan is selected, then the names of family members can be added, as well as an address and subscription service; and
once a plan is selected and all information entered, the final payment screen is displayed where the user will add credit card or other payment information.

15. The method of claim 1, wherein
the user's request exam information is displayed and presented for review while the user waits for tele-connection with medical services professional.

16. The method of claim 15, wherein
while waiting for a medical services provider, the user can complete a medical history intake form or update any such form connected and stored across the platform with an electronic medical recorders provide;
the user is displayed a plurality of screens asking them to enter yes/no questions with respect to conditions, allergies, and other relevant questions; and
once the intake of additional medical information has been completed, the user is displayed a waiting room screen notifying them of their wait time and any additional pertinent information.

17. The method of claim 1, wherein
a video call within the app is display to a user if required by state law, which is then followed up by a SMS text notification when any prescriptions prescribed as a result of the appointment and have been filled by the selected pharmacy.

18. The method of claim 1, further comprising the following steps for a subsequent visit or usage of the platform after creating an account,
a user selects the family member that is to be seen by a medical service provider;
the user either selects a chief complaint from a list of menus or by entering an unspecified complaint and can upload one or more images if/as necessary for assistance in being evaluated;
the user is then offer the choice to select or confirm an existing pharmacy from either a map display or a list display of potential pharmacy options based on location;
a biometric login is presented to the user while the system searches for and ques an available doctor to fulfil the request;
while waiting for a doctor, the user can review all their previously entered information and selections to make changes or edits; and
once all previously information is confirmed and final, a static display is presented to the user while waiting for a medical professional

19. The method of claim 18, wherein
biometric confirmation of the requested exam can be provided either with touch identification or a facial identification; or
a standard phone number and password login may also be presented for verification, along with standard password recovery options.

20. The method of claim 1, further comprising the steps for the application flow for reviewing past exams and making document notes
the user selects a member of the user's account to review the selected user's exam records;
biometric confirmation of the requested exam can be provided either with touch identification or a facial identification; or
a standard phone number and password login may also be presented for verification, along with standard password recovery options;
a standard phone number and password login may also be presented for verification, along with standard password recovery options;
a user can simply select from a list of present past exams the exam they would like to review; and
once selected, the exam summary and any doctor notes will be displayed to the user.
